(19) United States
(12) Patent Application Publication
(10) Pub. No.: US 2007/0077296 A1
(43) Pub. Date: Apr. 5, 2007

(54) Pharmaceutical Preparation containing Meloxicam

(76) Inventors: Martin A. Folger, Ingelheim (DE); Stefan Henke, Kirchen (DE); Jens Schmalz, Hueffelsheim (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/532,952

(22) Filed: Sep. 19, 2006

(30) Foreign Application Priority Data
Sep. 30, 2005 (EP) 05109064

(51) Int. Cl. A61K 9/20; A61K 31/5415

(52) U.S. Cl. 424/464; 514/226.5

(57) ABSTRACT

The invention relates to novel solid formulations comprising as pharmaceutically active compound meloxicam and to processes for producing such solid formulations. The invention furthermore relates to a method for manufacturing a medicament for the prevention and/or treatment of pain, inflammation, fever, acute mastitis, diarrhoea, locomotive disorders, lameness, osteoarthritis, problems of mobility or respiratory complaints, wherein the solid formulations according to the invention are used.

### BACKGROUND OF THE INVENTION

1. Technical Field

The invention relates to the field of animal health. In particular, the invention relates to novel oral pharmaceutical compositions comprising as pharmaceutically active compound meloxicam.

2. Background Information

Meloxicam (4-hydroxy-2-methyl-N-(5-methyl-2-thiazolyl)-2H-1,2-benzothiazine-3-carboxamide-1,1-dioxide) is an active substance which belongs to the group of NSAID's (non-steroidal-antiinflammatory drugs). Meloxicam and the sodium and meglumine salt thereof (N-methyl-D-glucamine salt) are described in EP-A-0 002 482 (corresponding U.S. Pat. No. 4,233,299). EP-A-0 945 134 (corresponding U.S. No. Pat. 6,869,948) discloses the pH-dependent solubility characteristics of meloxicam and its salts, i.e. the sodium salt, the ammonium salt and the meglumine salt, in aqueous solution. According to this, meloxicam is an active substance which does hardly dissolve in water. The meloxicam salts, particularly the meglumine salt, exhibit improved solubility as the pH increases between 4 and 10, as shown in Table 1 of EP 0945134. WO 2004-037264 (corresponding Published U.S. Patent Application 20040180092) discloses a granulated form of meloxicam which can be administered to animals by mixing it into their drinking water or as a food supplement.

The problem underlying the present invention was to provide a meloxicam solid formulation voluntarily acceptable by mammalian subjects, especially small animals.

### BRIEF SUMMARY OF THE INVENTION

The invention relates to novel solid formulations comprising as pharmaceutically active compound meloxicam or a pharmaceutically acceptable salt thereof which is homogenously dispersed in a carrier and a flavor acceptable to small animals. Preferably, such solid formulations are granules or tablets. Most preferred is a tablet characterized in that the tablet consists of 1 mg, 2.5 mg, 5 mg or 10 mg meloxicam, and further consists of meglumine preferably in a molar ratio of 10:8 (meglumine:meloxicam), hydroxypropylmethyl cellulose, polyvidone, glucose, lactose, microcrystalline cellulose, croscarmellose sodium, artificial beef flavor and magnesium stearate.

The invention further relates to fluid-bed granulation processes for production of the solid formulations comprising the steps:
a) an aqueous solution of meloxicam, a salt forming agent such as meglumine and a binder or two binders as defined above is sprayed onto a solid carrier bed comprising one or several carriers and/or excipients and
b) the mixture of a) is dried and
c) the mixture of b) is sieved and de-agglomerated and
d) an outer phase consisting of a carrier, a carrier/disintegrant, a disintegrant, a flavour and optionally a flow regulator is added to the mixture of c) and
e) a lubricant is added to the mixture of d) and
f) the mixture of e) is blended for uniformity of granules to obtain final granules and/or
g) the final granules of f) are compressed to solid formulations.

Step g) is omitted if the solid formulation is a granule. If the solid formulation is a tablet, step g) is carried out.

Furthermore, the invention relates to a method of prevention and/or treatment of diseases wherein NSAID's, preferably meloxicam, have a therapeutic benefit, comprising administering to a mammal in need of such treatment a therapeutically effective amount of a solid formulation according to the invention as disclosed above.

Preferred is a method of prevention and/or treatment of pain, inflammation, fever, acute mastitis, diarrhoea, locomotive disorders, lameness, osteoarthritis, problems of mobility or respiratory complaints, preferably pain, inflammation or locomotive disorders, most preferably pain or inflammation, comprising administering to a mammal in need of such treatment a therapeutically effective amount of a solid formulation according to the invention as disclosed above.

Most preferably, the method comprises administering a tablet according to the invention, as defined above.

Furthermore, the invention relates to a method for manufacturing a medicament for the prevention and/or treatment of a disease selected from the group consisting of pain, inflammation, fever, acute mastitis, diarrhoea, locomotive disorders, lameness, osteoarthritis, problems of mobility or respiratory complaints, preferably pain, inflammation or locomotive disorders, most preferably pain or inflammation, characterised in that a solid formulation according to the invention is used. Preferably, the invention relates to a method for manufacturing a medicament for the prevention and/or treatment of a disease selected from the group consisting of pain, inflammation, fever, acute mastitis, diarrhoea, locomotive disorders, lameness, osteoarthritis, problems of mobility or respiratory complaints, preferably pain, inflammation or locomotive disorders, most preferably pain or inflammation, characterised in that a tablet consisting of 1 mg, 2.5 mg, 5 mg or 10 mg meloxicam and further consisting of meglumine preferably in a molar ratio of 10:8 to meloxicam, hydroxypropylmethyl cellulose, polyvidone, glucose, lactose, microcrystalline cellulose, croscarmellose sodium, artificial beef flavor, and magnesium stearate is used.

### BRIEF DESCRIPTION OF THE DRAWINGS

FIG. 1: Illustration of the basic top spray fluid bed process

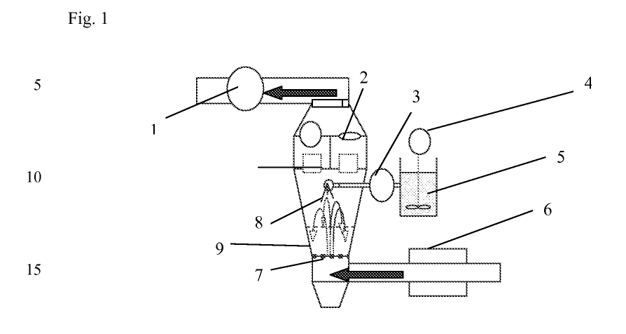

Reference signs:
1 Exhaust air ventilator; 2 Filter; 3 Pump; 4 Stirrer; 5 Aqueous Suspension of micronised meloxicam and binder solution (PVP, HPMC, starch, gelatine); 6 Heating device for inlet air; 7 Sieve; 8 Nozzle, aqueous suspension is sprayed onto powder bed (citric acid, lactose, starch, flavour); 9 Powder bed

FIG. 2: Flow Chart of Manufacturing Process

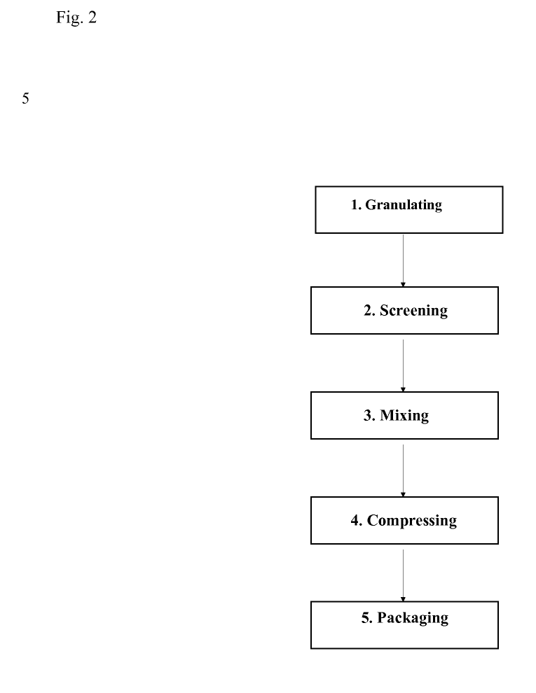

FIG. 3: Tablet disintegration data

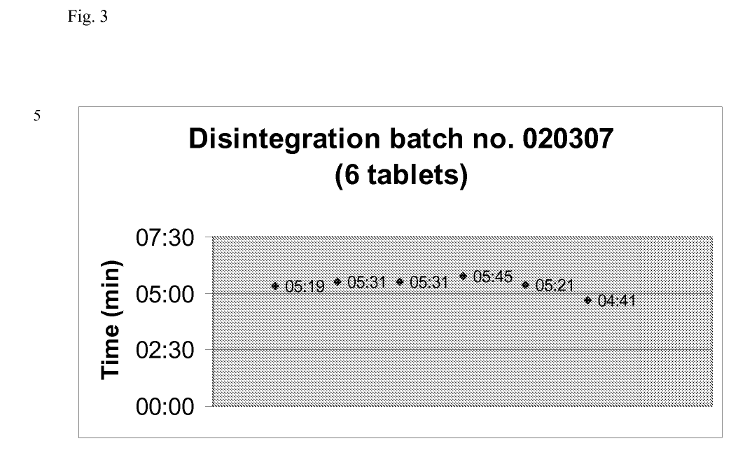

### DETAILED DESCRIPTION OF THE INVENTION

Definitions of Terms Used in the Description:

Before the embodiments of the present invention it must be noted that as used herein and in the appended claims, the singular forms “a ”, “an”, and “the” include plural reference unless the context clearly dictates otherwise. Thus, for example, reference to “a tablet” includes a plurality of such tablets, reference to the “carrier” is a reference to one or more carriers and equivalents thereof known to those skilled in the art, and so forth. Unless defined otherwise, all technical and scientific terms used herein have the same meanings as commonly understood by one of ordinary skill in the art to which this invention belongs. All given ranges and values may vary by 1 to 5% unless indicated otherwise or known otherwise by the person skilled in the art, therefore, the term “about” was omitted from the description. Although any methods and materials similar or equivalent to those described herein can be used in the practice or testing of the present invention, the preferred methods, devices, and materials are now described. All publications mentioned herein are incorporated herein by reference for the purpose of describing and disclosing the substances, excipients, carriers, and methodologies as reported in the publications which might be used in connection with the invention. Nothing herein is to be construed as an admission that the invention is not entitled to antedate such disclosure by virtue of prior invention.

The solution to the above technical problem is achieved by the description and the embodiments characterized in the claims.

To overcome the difficulties in the art, a process was invented. Only the invention of this novel fluid-bed granulation process allowed the formulation of voluntarily acceptable solid formulations according to the invention. With the process according to the invention, it was possible to formulate a voluntarily accepted, long-term stable, large scale producable, homogenously dispersed, fast-releasing solid formulation. Such solid formulations comprising a flavor suitable for small animals, which surprisingly still allows a formulation comprising meloxicam as a salt in a very low concentration in the formulation and yet have an excellent palatability. Thus, the solid formulations according to the invention are a major step forward in therapeutic application as they do not have to be force-fed to the animal.

In a first important embodiment, the invention relates to a solid formulation, comprising meloxicam or a pharmaceutically acceptable salt, preferably the meglumine salt, thereof, which is homogenously dispersed in a granulated carrier, and a flavor acceptable to small animals. Such flavors according to the invention preferably are selected from artificial beef flavours, artificical chicken flavours, pork liver extract, artificial meat flavour, honey flavour. Said flavors not only disguise the taste of the salt forming agent and other excipients, but also of meloxicam.

Preferably, the solid formulation according to the invention is a tablet or granule formulation. The granule formulation according to the invention is explained in more detail below. More preferably, the solid formulation is chewable.

The invention preferably also relates to a solid formulation according to the invention, further comprising one or several pharmaceutically acceptable excipients.

Excipients according to the invention are preferably selected from the group consisting of diluents, disintegrants, carriers, binders, flow regulators, lubricants and solvents. Any other excipient known to the skilled person and found suitable for the solid formulation according to the invention may also be comprised in the solid formulation according to the invention. See also Remington, J. P. The science and Practice of Pharmacy (2000). 20th ed. Lippincott Williams & Wilkins Publishers, Philiadelphia, US.

More preferably, said excipients are carriers/disintegrants selected from the group lactose, starch, sugars, e.g. glucose and/or sugar alcohols, e.g. sorbitol, cellulose, microcrystalline cellulose and cellulose derivatives, e.g. methylcellulose. Any other carrier known to the skilled person and found suitable for the solid formulation according to the invention may also be comprised in the solid formulation according to the invention. See also Remington, J. P. The science and Practice of Pharmacy (2000). 20th ed. Lippincott Williams & Wilkins Publishers, Philiadelphia, US.

One or several binders according to the invention are preferably selected from the group consisting of polyvidone (used synonymously for povidone), methylcellulose, hydroxypropylmethylcellulose (HPMC), hydroxymethylcellulose, starch, and gelatine. Any other binder known to the skilled person and found suitable for the solid formulation according to the invention may also be comprised in the solid formulation according to the invention. See also Remington, J. P. The science and Practice of Pharmacy (loc. cit.).

The solid formulation according to the invention may also comprise one or several flow regulators selected from the group consisting of silica, preferably colloidal anhydrous silica, calcium silicate, magnesium silicate and talc. Any other flow regulator known to the skilled person and found suitable for the solid formulation according to the invention may also be comprised in the solid formulation according to the invention. See also Remington, J. P. The science and Practice of Pharmacy (loc. cit.).

The solid formulation according to the invention may also comprise one or several disintegrants selected from the group consisting of croscarmellose sodium, sodium starch glycolate, pregelatinised starch and cross-linked polyvinylpyrrolidone. Any other disintegrant known to the skilled person and found suitable for the solid formulation according to the invention may also be comprised in the solid formulation according to the invention. See also Remington, J. P. The science and Practice of Pharmacy (loc. cit.).

The solid formulation according to the invention may also comprise one or several lubricants selected from the group consisting of magnesium stearate, calcium stearate, glyceryl behenate, polyethylene glycol, stearic acid and talc. Any other lubricant known to the skilled person and found suitable for the solid formulation according to the invention may also be comprised in the solid formulation according to the invention. See also Remington, J. P. The science and Practice of Pharmacy (loc. cit.).

The invention preferably also relates to a solid formulation according to the invention, characterized in that the carriers are glucose. The invention preferably also relates to a solid formulation according to the invention, characterized in that the lactose consists of particles which have been spray-dried in order to improve compression characteristics. The person skilled in the art knows other types of lactose which are suitable as well as carrier according to the invention, e.g. fine lactose equal or smaller than 200 μm in size or coarse lactose with particles bigger than 200 μm in size. lactose. Preferred is spray-dried lactose.

The invention preferably also relates to a solid formulation according to the invention, characterized in that the starch or various starches are selected from the group consisting of native starch, gelatinized starch, partly gelatinized starch, starch powder, starch granules, chemically modified starch and swellable physically modified starch.

The invention preferably also relates to a solid formulation according to the invention, comprising 0.5 to 20 mg of meloxicam. The more preferred solid formulation contains 1 to 10 mg of meloxicam. The even more preferred solid formulation contains 1 to 5 mg of meloxicam. Most preferred solid formulations contain 1 mg, 2.5 mg, 5 mg or 10 mg of meloxicam.

The invention preferably also relates to a solid formulation according to the invention, comprising a content of 8:8-8:12 of meloxicam in relation to meglumine, preferably 8:10.

The invention preferably also relates to a solid formulation according to the invention, characterized in that the weight of the whole solid formulation is in the range of 150 to 3000 mg, with a more preferred weight range of 150 mg to 2000 mg, and most preferred weight of 200 mg, 500 mg, 1000 mg or 2000 mg.

The invention preferably also relates to a solid formulation according to the invention, characterized in that the solid formulation is produced by a fluid-bed granulation process comprising the steps:
a) an aqueous solution of meloxicam, a salt forming agent such as meglumine and a binder or two binders as defined above is sprayed onto a solid carrier bed comprising one or several carriers and/or excipients and
b) the mixture of a) is dried and
c) the mixture of b) is sieved and de-agglomerated and
d) an outer phase consisting of a carrier, a carrier/disintegrant, a disintegrant, a flavour and optionally a flow regulator is added to the mixture of c) and
e) a lubricant is added to the mixture of d) and
f) the mixture of e) is blended for uniformity of granules to obtain final granules and/or
g) the final granules of f) are compressed to solid formulations.

Step g) is omitted if the solid formulation is a granule. If the solid formulation is a tablet, step g) is carried out.

The invention preferably also relates to a solid formulation according to the invention, characterized in that the solid formulation is produced by a fluid-bed granulation process comprising the steps:
a) an aqueous solution of meloxicam, meglumine, hydroxypropylmethyl cellulose and povidone is sprayed onto a solid carrier bed comprising glucose monohydrate and
b) the mixture of a) is dried and
c) the mixture of b) is sieved and de-agglomerated and
d) an outer phase consisting of one or more suitable flavours, one or more suitable carriers and one or more suitable disintegrants, is added to the mixture of c) and
e) a lubricant is added to the mixture of d) and
f) the mixture of e) is blended for uniformity of granules to obtain final granules and/or
g) the final granules of f) are compressed to solid formulations.

Step g) is omitted if the solid formulation is a granule. If the solid formulation is a tablet, step g) is carried out.

The invention preferably relates to a granule formulation as obtained by the process above that can either be administered in the granular form or as tablets after compressing the final granules to tablets. Therefore, the solid formulation according to the invention preferably is a granule (or a plurality of such granules) or a tablet. The administration of the granules can take place by mixing with food or by offering the granules directly to the animal, e.g. in a bowl. The application of the granular form will allow an individual dosing of meloxicam according to the body weight of the animal.

The tablets according to the invention have surprising advantages. The disintegration behaviour is ensuring immediate release of meloxicam. Surprisingly, it could be demonstrated that while compressing the final granules as mentioned above, a decrease in the disintegration characteristics is not observed. By ensuring an immediate release profile of meloxicam, the amount of drug to be administered can be kept as low as possible, thereby improving the safety profile especially for long-term treatment. Furthermore, the dosing accuracy of the tablet is excellent. This is due to the fact that in accordance with the manufacturing process according to this invention, an excellent uniformity of meloxicam content is achieved. Furthermore, the tablets can be broken into two halves so that half the dose per tablet can be administered. This is even more important since the drug is administered for a life-long treatment.

Also, palatability of the tablet is excellent. Compared with the existing tablet formulation for human use, the compliance of both the animal and the animal owner are significantly improved. This is even more important since the drug is administered for a life-long treatment.

The invention preferably also relates to a tablet according to the invention, characterized in that the tablet is stable at 25° C./60% relative humidity. In the examples, testing parameter assays are disclosed for disintegration of the tablet.

Suitable packaging materials for tablets according to the invention are selected from, but not limited to: aluminum/aluminum blisters, PVC/PVDC blisters, and HDPE (high density polyethylene bottles).

The invention preferably relates to a solid formulation, and most preferred a tablet according to the invention, characterized in that the solid formulation or tablet consists of 1 mg, 2.5 mg, 5 mg or 10 mg meloxicam, and further consists of meglumine preferably in a molar ratio of 8:8 to 12:8, especially preferably in a molar ratio of 10:8 (meglumine:meloxicam), hydroxypropylmethyl cellulose (0-5%), polyvidone (0-5%), glucose (20-60%), lactose (10-40%), microcrystalline cellulose (10-30), croscarmellose sodium (1-7%), artificial beef flavor (2-20%), and magnesium stearate (0.25-2%).

In another important embodiment, the invention relates to a fluid-bed granulation process comprising the steps:
a) an aqueous solution of meloxicam, meglumine, and one or two binders is sprayed onto a solid carrier bed comprising glucose monohydrate and
b) the mixture of a) is dried and
c) the mixture of b) is sieved and de-agglomerated and
d) an outer phase consisting of one or more suitable flavours, one or more suitable carriers and one or more suitable disintegrants, is added to the mixture of c) and
e) a lubricant is added to the mixture of d) and
f) the mixture of e) is blended for uniformity of granules to obtain final granules and/or
g) the final granules of f) are compressed to solid formulations.

Step g) is omitted if the solid formulation is a granule. If the solid formulation is a tablet, step g) is carried out.

The invention preferably relates to a fluid-bed granulation process comprising the steps:
a) an aqueous solution of meloxicam, meglumine, hydroxypropylmethyl cellulose and povidone is sprayed onto a solid carrier bed comprising glucose monohydrate and
b) the mixture of a) is dried and
c) the mixture of b) is sieved and de-agglomerated and
d) an outer phase consisting of one or more suitable flavours, one or more suitable carriers and one or more suitable disintegrants, is added to the mixture of c) and
e) a lubricant is added to the mixture of d) and
f) the mixture of e) is blended for uniformity of granules to obtain final granules and/or
g) the final granules of f) are compressed to solid formulations.

Step g) is omitted if the solid formulation is a granule. If the solid formulation is a tablet, step g) is carried out.

Another embodiment is a method of prevention and/or treatment of diseases wherein substances for the prevention and/or treatment of a disease selected from the group consisting of pain, inflammation, fever, acute mastitis, diarrhoea, locomotive disorders, lameness, osteoarthritis, problems of mobility or respiratory complaints, characterised in that a solid formulation according to the invention is used, comprising administering to a mammal in need of such treatment a therapeutically effective amount of a solid formulation according to the invention as disclosed above.

Preferred is a method of prevention and/or treatment of a disease selected from the group consisting of pain, inflammation, fever, acute mastitis, diarrhoea, locomotive disorders, lameness, osteoarthritis, problems of mobility or respiratory complaints, preferably pain, inflammation or locomotive disorders, most preferably pain or inflammation, comprising administering to a mammal in need of such treatment a therapeutically effective amount of a solid formulation according to the invention as disclosed above.

Most preferably, the method comprises administering a tablet according to the invention, characterized in that the tablet consists of 1 mg, 2.5 mg, 5 mg or 10 mg meloxicam, and further consists of meglumine preferably in a molar ratio of 10:8 to meloxicam, hydroxypropylmethyl cellulose, polyvidone, glucose, lactose, microcrystalline cellulose, croscarmellose sodium, artificial beef flavor, and magnesium stearate.

Preferably also, such treatment is by orally applying the solid formulation according to the invention.

The mammal according to the invention is preferably a mammal selected from the group consisting of dogs, cats and rodents such as rabbits.

Furthermore, the invention relates to a method for manufacturing a medicament for the prevention and/or treatment of of a disease selected from the group consisting of pain, inflammation, fever, acute mastitis, diarrhoea, locomotive disorders, lameness, osteoarthritis, problems of mobility or respiratory complaints, preferably pain, inflammation or locomotive disorders, characterised in that a solid formulation according to the invention is used. Preferably, the invention relates to a method for manufacturing a medicament for the prevention and/or treatment of pain, inflammation, fever, acute mastitis, diarrhoea, locomotive disorders, lameness, osteoarthritis, problems of mobility or respiratory complaints, characterised in that a tablet consisting of 1 mg, 2.5 mg, 5 mg or 10 mg meloxicam and further consisting of meglumine preferably in a molar ratio of 10:8 to meloxicam, hydroxypropylmethyl cellulose, polyvidone, glucose, lactose, microcrystalline cellulose, croscarmellose sodium, artificial beef flavor, and magnesium stearate is used. The following examples serve to further illustrate the present invention; but the same should not be construed as limiting the scope of the invention disclosed herein.

### EXAMPLE 1

### Compositions

A)

| | | mg/tablet |
|---|---|---|
| | | 1.5 mg |
| | Ingredients | chewable |
| (01) | Meloxicam | 1.50 |
| (02) | Meglumine | 1.05 |
| (03) | Hydroxypropylmethyl | 7.50 |
| | cellulose | |
| (04) | Polyvidon | 5.00 |
| (05) | Glucose monohydrate | 234.95 |
| (06) | Spray-dried lactose | 105.00 |
| (07) | Microcrystalline | 70.00 |
| | cellulose | |
| (08) | Croscarmellose sodium | 20.00 |
| (09) | Artificial Beef Flavour | 50.00 |
| (10) | Magnesium stearate | 5.00 |
| (11) | Purified water as | |
| | volatile ingredient | |
| | | 500.000 |

B)

| | mg/tablet | mg/tablet | mg/tablet | mg/tablet |
|---|---|---|---|---|
| | 1 mg | 2.5 mg | 5 mg | 10 mg |
| Ingredients | chewable | chewable | chewable | chewable |
| Meloxicam | 1.00 | 2.50 | 5.00 | 10.00 |
| Meglumin | 0.70 | 1.75 | 3.50 | 7.00 |
| Hydroxypropylmethyl | 3.00 | 7.50 | 15.00 | 30.00 |
| cellulose | | | | |
| Polyvidon | 2.00 | 5.00 | 10.00 | 20.00 |
| Glucose monohydrate | 93.30 | 233.25 | 466.50 | 933.00 |
| Spray-dried lactose | 42.00 | 105.00 | 210.00 | 420.00 |
| Microcrystalline | 28.00 | 70.00 | 140.00 | 280.00 |
| cellulose | | | | |
| Croscarmellose sodium | 8.00 | 20.00 | 40.00 | 80.00 |
| Artificial Beef flavour | 20.00 | 50.00 | 100.00 | 200.00 |
| Magnesium stearate | 2.00 | 5.00 | 10.00 | 20.00 |
| Purified water as | | | | |
| volatile ingredient | | | | |
| | 200.00 | 500.00 | 1000.00 | 2000.00 |

### EXAMPLE 2

### Raw Materials

(01) Meloxicam
   Function: Active ingredient
(02) Meglumine
   Function: Salt-forming agent
(03) Hydroxypropylmethyl cellulose
   Function: Binder
(04) Povidone
   Function: Binder
(05) Glucose monohydrate
   Function: Carrier
(06) Lactose, spray-dried
   Function: Diluent, Disintegrant
(07) Microcrystalline cellulose
   Function: Diluent, Disintegrant
(08) Croscarmellose sodium
   Function: Disintegrant
(09) Artificial Beef Flavour
   Function: Flavour
(10) Magnesium stearate
   Function: Lubricant
(11) Purified water
   Function: Solvent

### EXAMPLE 3

### Manufacturing Process

1 batch=350000 tablets (1 mg Dosage)
1 batch=140000 tablets (2.5 mg Dosage)
1 batch=70000 tablets (5 mg Dosage)

1 batch=35000 tablets (10 mg Dosage)

| | | |
|---|---|---|
| 1. | Granulating | |
| | Transfer in a suitable Granulator after prescreening: | in kg |
| (01) | Glucose monohydrate | 32.655 |
| (02) | Meglumine (Spray solution) | 0.245 |
| (03) | Meloxicam (Spray solution) | 0.350 |
| (04) | Povidone (Spray solution) | 0.700 |
| (05) | Hydroxypropylmethylcellulose | 1.05 |
| | Premix in the granulator and granulate | 35.000 |
| | Purified water is used as a solvent for | 10-22 |
| | the spray solution of meloxicam, | |
| | meglumine, povidone and | |
| | hydroxypropylmethylcellulose. | |
| | After completion of the spraying step | |
| | the granules are dried. | |
| 2. | Screening | |
| | Screen the premixture (1.). | 35.000 kg |
| 3. | Final mixing | |
| | Add | |
| (06) | Lactose, spray-dried | 14.700 kg |
| (07) | Microcrystalline cellulose | 9.800 kg |
| (08) | Croscarmellose sodium | 2.800 kg |
| (09) | Artificial Beef Flavour | 7.000 kg |
| (10) | Magnesium stearate | 0.700 kg |
| | In a tumbling mixer, mix the | |
| | screened premixture (2.) and the five ingredients ad | 70.000 kg |
| 4. | Compression | |
| | Using a rotary press, compress the | |
| | final mixture (3.) | 70.000 kg |
| | into tablets of 200 mg, 500 mg, 1000 mg, 2000 mg. | |
| 5. | Packaging | |
| | Transfer the tablets in a suitable container. | |
| | The tablets can be packed e.g. by blistering of the | |
| | tablets in a suitable machine. | |

1. A solid formulation, comprising meloxicam or a pharmaceutically acceptable salt thereof which is homogenously dispersed in a granulate carrier, and a flavour suitable for small animals.

2. A solid formulation according to claim 1, further comprising pharmaceutically acceptable carriers and/or excipients.

3. A solid formulation according to claim 2, characterized in that the carriers and/or excipients are selected from the group consisting of diluents, disintegrants, carriers, binders, flavours, flow regulators, lubricants and solvents.

4. A solid formulation according to claim 2, characterized in that the carriers are glucose, lactose, and microcrystalline cellulose.

5. A solid formulation according claim 4, wherein the lactose is spray-dried lactose.

6. A solid formulation according to claim 1, comprising 0.5 to 20 mg of meloxicam.

7. A solid formulation according to claim 1, wherein the solid formulation is a tablet or a granule.

8. A fluid-bed granulation process comprising the steps:
a) an aqueous solution of meloxicam, meglumine, and one or two suitable binders is sprayed onto a solid carrier bed comprising one or more suitable carriers and
b) the mixture of a) is dried and
c) the mixture of b) is sieved and de-agglomerated and
d) an outer phase consisting of one or more suitable flavours, one or more suitable carriers and one or more suitable disintegrants, is added to the mixture of c) and
e) a lubricant is added to the mixture of d) and
f) the mixture of e) is blended for uniformity of granules to obtain final granules.

9. The process of claim 8, further comprising a step g) wherein the final granules of f) are compressed to solid formulations.

10. A fluid-bed granulation process according to claim 8, comprising the steps:
a) an aqueous solution of meloxicam, meglumine, hydroxypropylmethyl cellulose and polvidone is sprayed onto a solid support comprising glucose and
b) the mixture of a) is dried and
c) the mixture of b) is sieved and de-agglomerated and
d) an outer phase consisting of one or more suitable flavours, one or more suitable carriers and one or more suitable disintegrants, is added to the mixture of a flow regulator is added to the mixture of c) and
e) a lubricant is added to the mixture of d) and
f) the mixture of e) is blended for uniformity of granules to obtain final granules.

11. The process of claim 10, further comprising a step g) wherein the final granules of f) are tabletted.

12. A method for preventing and/or treating pain, inflammation or locomotive disorders, comprising administering to an individual a solid formulation according to any one of claims 1 to 7.
